(19) United States
(12) Patent Application Publication
(10) Pub. No.: US 2023/0226411 A1
(43) Pub. Date: Jul. 20, 2023

(54) SPEED GENERATION METHOD FOR SIMULATING RIDING

(71) Applicant: UNIWILL TECHNOLOGY INC., Taipei (TW)

(72) Inventors: Chun-Cheng CHEN, Taipei (TW); Chun-Ting LIU, Taipei (TW); Sung-Yi CHUANG, Taipei (TW)

(21) Appl. No.: 18/076,277

(22) Filed: Dec. 6, 2022

(30) Foreign Application Priority Data
Jan. 17, 2022 (TW) 111101877

(51) Int. Cl. A63B 24/00; A63B 22/06

(52) U.S. Cl. CPC A63B 24/0062; A63B 22/0605; A63B 2024/0093; A63B 2071/0644; A63B 2220/30; A63B 2220/833; A63B 2225/50

(57) ABSTRACT

A speed generation method for simulating riding is provided. A computer device (20) retrieves a riding weight and road condition information, selects one of a plurality of preset gear ratios (30) as a selected gear ratio, retrieves a wheel rotating speed of a bicycle (4), and determines a simulated speed in a simulating riding service based on the selected gear ratio, a tire parameter, and the rotating speed.

### BACKGROUND OF THE DISCLOSURE

### Technical Field

The disclosure generally relates to simulating riding, and more particularly, to a speed generation method for simulating riding.

### Description of Related Art

Multiple kinds of simulating riding services are provided, so the users can experience indoor riding as if they ride outdoors as the first person themselves.

The speed generation method for the simulating riding service retrieves, by the pedal sensor, the cycling speed that the user pedals and computes the simulated speed of the bicycle in the simulating riding service based on the cycling speed.

The speed generation method does not nevertheless consider the bicycle derailleur. That is, the simulated speed of the bicycle is computed only by a fixed ratio and the cycling speed regardless of the routes and the road condition (such as the uphill, the level ground, and the downhill).

Therefore, the user has no chance to challenge the riding with different routes or road conditions in the simulating riding service, so the user experience is worse.

Another speed generation method for a simulating riding service simulates a resistance force based on real road conditions implemented by a professional smart trainer and computes the simulated speed of the simulating riding service according to the pedaling power when the user rides the bicycle.

The speed generation method can retrieve the simulated speed; however, the professional smart trainer is necessary and expensive. Therefore, the simulating riding service implemented by the speed generation method is hard to become popular.

Accordingly, the problems with the speed generation method raised above should be solved by a more effective solution.

### SUMMARY OF THE DISCLOSURE

The present disclosure is directed to a speed generation method for simulating riding to simulate a simulated speed matching a user’s requirement by dynamically changing a gear ratio without equipping any professional smart trainer.

In one embodiment, a speed generation method for simulating riding includes: a) retrieving, by a computer device, a riding weight and road condition information of a simulating riding service; b) selecting one of a plurality of preset gear ratios as a selected gear ratio based on the riding weight and the road condition information, wherein the plurality of preset gear ratios respectively correspond to a plurality of weight conditions and a plurality of road conditions; c) retrieving a wheel rotating speed of a bicycle; and d) determining a simulated speed based on the selected gear ratio, a tire parameter, and the wheel rotating speed.

Accordingly, the present disclosure may dynamically change the gear ratio based on the riding weight and the road condition and simulate a variable speed effect in the simulating riding service to increase the user experience without the professional smart trainer.

### DESCRIPTION OF THE DRAWINGS

FIG. 1 is a diagram for a speed generation method of the related art.

FIG. 2 is a diagram illustrating a simulating riding system of the present disclosure.

FIG. 3 is a flowchart illustrating a speed generation method according to one embodiment of the present disclosure.

FIG. 4 is a flowchart illustrating a speed generation method according to one embodiment of the present disclosure.

FIG. 5 is a part of flowchart illustrating a speed generation method according to one embodiment of the present disclosure.

FIG. 6 is a part of flowchart illustrating a speed generation method according to one embodiment of the present disclosure.

FIG. 7 is a schematic diagram illustrating the simulating riding system according to one embodiment of the present disclosure.

FIG. 8 is a diagram illustrating a distribution of a gear ratio and a thrust force ratio according to one embodiment of the present disclosure.

FIG. 9 is a diagram illustrating a gear-ratio lookup table according to one embodiment of the present disclosure.

### DETAILED DESCRIPTION

In cooperation with the attached drawings, the technical contents and detailed description of the present disclosure are described hereinafter according to multiple embodiments, being not used to limit its executing scope. Any equivalent variation and modification made according to appended claims is all covered by the claims claimed by the present disclosure.

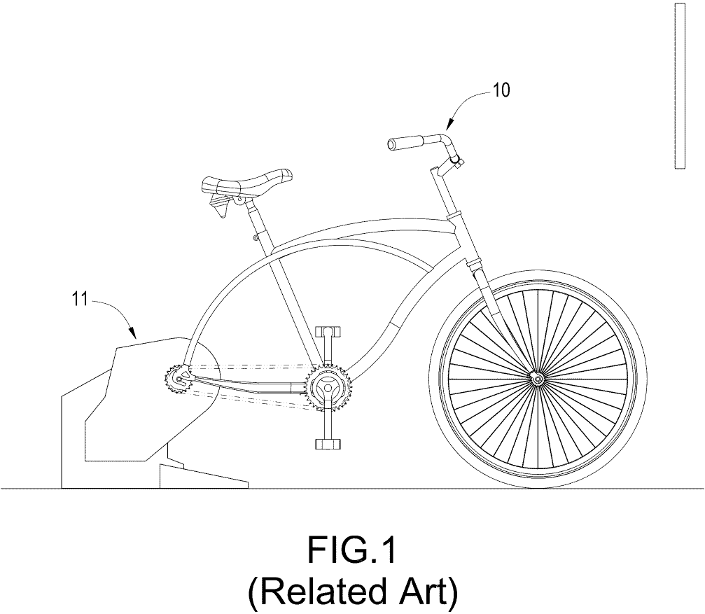

Please refers to FIG. 1. FIG. 1 is a diagram of a speed generation method of the related art. The speed generation method of the related art requires a professional and expensive smart trainer 11 simulating a resistance of an actual road condition, and the user may change a gear ratio of a bicycle 10 manually according to the resistance.

Specifically, before a simulating riding service is utilized, the user has to install the bicycle 10 on the smart trainer 11.

In the installation process, the user has to take a rear wheel and a freewheel (a rear gear wheel plate) apart from the bicycle 10 and put a chain of the bicycle 10 onto a freewheel of the smart trainer 11.

Accordingly, the user may control a gear shifting by the freewheel of the smart trainer 11. Furthermore, the smart trainer 11 may sense a pedaling of the user to generate a power value and compute a simulated speed based on the power value in real-time.

The speed generation method of the related art induces the drawbacks: (1) the price of the smart trainer 11 is high so that the user lacks intention of buying the smart trainer 11; (2) the installation process of the smart trainer 11 is complicated, and the user must spend a lot of time installing/dismantling the smart trainer 11 when/after applying the simulating riding service, such that the user lacks intention to use the simulating riding service.

For solving the problem described above, the present disclosure provides a novel simulating riding system and a speed generation method and may dynamically change the gear ratio based on a data statistic and analysis to implement a dynamical gear shifting in the simulating riding service. Therefore, the user may experience simulating riding with the variable speed close to real circumstances.

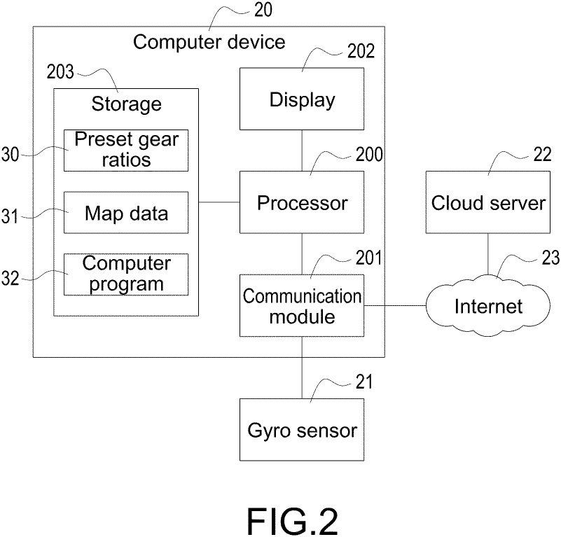

Please refers to FIG. 1 and FIG. 2. FIG. 2 is a diagram illustrating a simulating riding system of the present disclosure.

In one embodiment, the simulating riding system 2 includes a computer device 20, a gyro sensor 21, and a cloud server 22.

The computer device 20 (e.g., the smartphone, the smart television, the tablet computer, the notebook, or the personal computer, etc.) includes a communication module 201, a display 202, a storage 203, and a processor 200 which is electrically connected with the communication module 201, the display 202, and the storage 203.

The communication module 201 is configured to communicate with external devices. For example, the communication module 201 communicates with the cloud server 22 to transmit data through the Internet 23 and/or connects with the gyro sensor 21 through a wired connection or a wireless connection to retrieve data (e.g., a wheel rotating speed described later) from the gyro sensor 21.

The wired connection includes, for example but is not limited to, the USB cable, the Ethernet cable, or other types of cables. The wireless connection includes, for example but is not limited to, the Bluetooth connection, the Wi-Fi connection, the ZigBee connection, or other types of connection.

The display 202 (such as a built-in display or an external display) is configured to display screen images, such as displaying a screen image of executing the simulating riding service.

The storage 203 is configured to store data, such as one or more preset gear ratios 30, one or more map data 31, and computer program 32 for the simulating riding service.

The processor 200 is configured to control the computer device 20, such as executing the computer program 32.

The cloud server 22 is configured to provide the simulating riding service and to provide related data of the simulating riding service (such as the preset gear ratio 30, the map data 31, and/or the computer program 32) to the computer device 20.

In one embodiment, the user may operate the computer device 20 to download the computer program 32 (e.g., the simulating riding service App) through the Internet 23 and operate the computer device 20 to execute the computer program 32.

When executing the computer program 32, the computer device 20 may connect to the cloud server 22 to download the preset gear ratio 30 and the map data 31. Furthermore, after downloading the preset gear ratio 30 and the map data 31, the computer device 20 may select the preset gear ratio 30 and the map data 31 to start the simulating riding service.

In one embodiment, the gyro sensor 21 may be a pedaling frequency sensor of the bicycle. The pedaling frequency sensor is disposed on pedals of the bicycle before the simulating riding service is started, so the pedaling frequency sensor may sense a pedaling frequency of the bicycle (corresponding to the wheel rotating speed of the bicycle) in the simulating riding service.

In one embodiment, the gyro sensor 21 includes at least one of an acceleration meter, a gyro, a pressure sensor, a press switch, and other types of motion sensors, so the pedaling frequency may be computed by an acceleration variation, a direction variation, a pressure variation, a switch status, or other motion status monitored, and the wheel rotating speed is obtained.

In one embodiment, the gyro sensor 21 may be the smart pedaling frequency sensor produced by Bryton company or the Cadence Sensor produced by Garmin company but the present disclosure is not limited to.

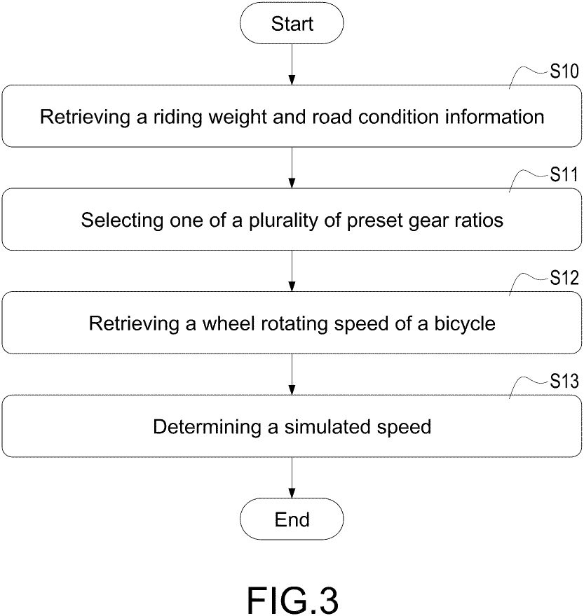

Please refers to FIG. 1 to FIG. 3. FIG. 3 is a flowchart illustrating a speed generation method according to one embodiment of the present disclosure. The speed generation method may be implemented by the simulating riding system 2 in each embodiment of the present disclosure.

In one embodiment, the gear ratio (such as a preset gear ratio or a selected gear ratio) may be defined dividing the teeth number of the chainring (the front gear wheel) by the teeth number of the freewheel (the rear gear wheel). That is, the gear ratio is the ratio between the teeth numbers of the two gears.

In other words, when the gear ratio is large (i.e., high gear ratio, such as more teeth number of the chainring and/or fewer teeth number of the freewheel), the user has to push more force on foot, whereas high speed may be obtained with the same wheel rotating speed. When the gear ratio is small (i.e., low gear ratio, such as fewer teeth number of the chainring and/or more teeth number of the freewheel), the user pushes less force on foot, whereas low speed may be obtained with the same wheel rotating speed.

In step S10, the computer device 20 may retrieve a riding weight and road condition information of the simulating riding service.

The riding weight and the road condition information may be retrieved by being inputted by the user manually or automatically retrieved by the simulating riding service (such as setting based on the bicycle and the body shape of the user and/or a selected riding route) but is not limited herein.

The riding weight is applied to reflect a resistance force induced by the user and the bicycle in the riding, so the riding weight may be one reference factor for selecting the most suitable gear ratio.

For example, when the riding weight is large, the resistance force is also large and the gear ratio for easily pedaling may be selected; when the riding weight is small, the resistance force is also small and the gear ratio for higher speed may be selected.

In one embodiment, the riding weight may be the weight of the bicycle, the weight of the user, or the total weight of the bicycle and the user.

The road condition information is applied to reflect a circumstance resistance in the riding, so the road condition information may be another reference factor for selecting the most suitable gear ratio.

For example, when the road is rugged or muddy, the gear ratio for easily pedaling may be selected; when the road is flat, the gear ratio for higher speed may be selected.

For another example, when the road slope is steep, the gear ratio for easily pedaling may be selected; when the road slope is smooth, the gear ratio for higher speed may be selected.

In step S11, the computer device 20 may select one of a plurality of preset gear ratios 30 as the selected gear ratio based on the riding weight and the road condition information.

In one embodiment, the computer device 20 may download the plurality of preset gear ratios 30 from the cloud server 22 in advance. The plurality of preset gear ratios 30 respectively correspond to multiple weight conditions and multiple road conditions.

The computer device 20 may select a weight condition matched according to a current riding weight, select a road condition matched according to a current road condition information, and correspondingly selects one of the preset gear ratios 30 as the selected gear ratio according to the selected weight condition and the selected road condition.

In one embodiment, the road condition information includes slope information. The plurality of road conditions includes a plurality of slope ranges. The computer device 20 may select one of the preset gear ratios 30 that has the slope range matching the slope information as the selected gear ratio.

In one embodiment, the plurality of weight conditions includes a plurality of weight ranges. The computer device 20 may select one of the preset gear ratios 30 that has the weight range matching the riding weight as the selected gear ratio.

In one embodiment, the computer device 20 may select one of the preset gear ratios 30 that has the slope range matching the slop information and the weight range matching the riding weight as the selected gear ratio.

In one embodiment, the plurality of preset gear ratios 30 are recorded in a lookup table.

The lookup table may be downloaded from the cloud server 22 and may record corresponding relations among the plurality of weight conditions, the plurality of road conditions, and the plurality of preset gear ratios.

In step S12, the computer device 20 may retrieve the wheel rotating speed of the bicycle through the gyro sensor 21.

In one embodiment, the wheel rotating speed is the cycling number of the pedal of the bicycle in unit time (such as in one minute or 10 seconds).

In step S13, the computer device 20 may retrieve a tire parameter of the bicycle, thus the computer device 20 may determine a current simulated speed of the simulating riding service based on the selected gear ratio, the tire parameter, and the wheel rotating speed.

In one embodiment, the tire parameter of the bicycle may be retrieved by being inputted by the user manually or automatically retrieved by the simulating riding service (such as setting based on the bicycle data from the user), but is not limited herein.

In one embodiment, the tire parameter may include a tire diameter and/or a tire width of a rear wheel. The computer device 20 may determine the simulated speed based on the selected gear ratio, the wheel rotating speed, and at least one of the tire diameter and the tire width.

In one embodiment, the computer device 20 may compute the simulated speed by the formula (1):

$$\begin{matrix}\text{The} \text{simulated} \text{speed} = \\ \text{π} \times \left(\text{the} \text{tire} \text{diameter} + \left(2 \times \text{the} \text{tire} \left(\left(\text{width}\right)\right)\right)\right) \times \\ \left(\text{the} \text{selected} \text{gear}\right) \left(\text{ratio}\right) \times \text{the} \text{wheel} \text{rotating} \text{speed}\end{matrix}$$

Accordingly, the present disclosure may automatically retrieve a suitable gear ratio based on the current weight and the road condition and simulate the variable speed effect in the simulating riding service to increase the user experience without the professional smart trainer.

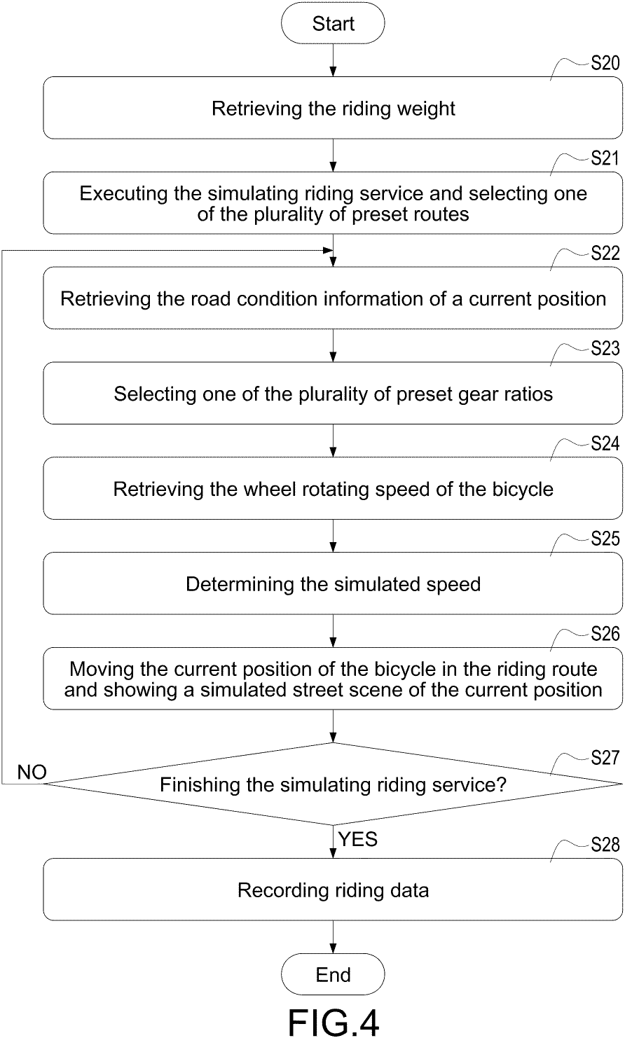

Please refers to FIG. 1 to FIG. 4. FIG. 4 is a flowchart illustrating a speed generation method according to one embodiment of the present disclosure. In one embodiment, the speed generation method includes steps S20-S28.

In step S20, the computer device 20 retrieves the riding weight.

In one embodiment, the riding weight may be retrieved by being inputted manually through a human-machine interface (not shown in the Figs.) of the computer device 20 by the user or automatically retrieved by the computer device 20 from user data.

In step S21, the computer device 20 executes the simulating riding service and selects one of the pluralities of preset routes as the riding route according to operations.

In one embodiment, the plurality of map data 31 respectively correspond to the plurality of preset routes and record 3D street scene corresponding to the preset routes. The user may select one of the pluralities of map data 31 through the human-machine interface of the computer device 20 and set the preset route of the selected map data 31 as the riding route.

In step S22, the computer device 20 retrieves a current position (with the road condition information) of the riding route of the bicycle in the simulating riding service.

In one embodiment, the computer device 20 may search information such as a slope, a smoothness of the road, and the like of the current position in the map data 31 to be (the road condition information of) the current position.

Steps S23-S25 are executed to retrieve the simulated speed. Steps S23-S25 are similar to steps S11-S13 and the description is not repeated.

In step S26, the computer device 20 may move the current position of the bicycle in the riding route of the selected map data 31 in the simulating riding service based on the retrieved simulated speed and show a simulated street scene of the current position on the display 202.

Because the simulated street scene may change along with the simulated speed of the bicycle ridden by the user, the user may enj oy a good experience in the simulating riding service as if the user rides in real circumstances.

In step S27, the computer device 20 determines whether the simulating riding service is finished, such as finishing riding, stopping the simulating riding service, or turning off the computer device 20.

When the computer device 20 determines that the simulating riding service is finished, step S28 is processed.

When determining that the simulating riding service is not finished, the computer device 20 goes back to step S22.

In step S28, the computer device 20 may record riding data of the simulating riding this time, such as a selected gear ratio record, a wheel rotating speed record, a route record, a riding time record, a road condition record, a riding weight record, and so on.

The present disclosure may compute the simulated speed based on the wheel rotating speed and simulate the bicycle speed with reasonable physical movement in the simulating riding service through only utilizing the low-price and durable gyro sensor 21.

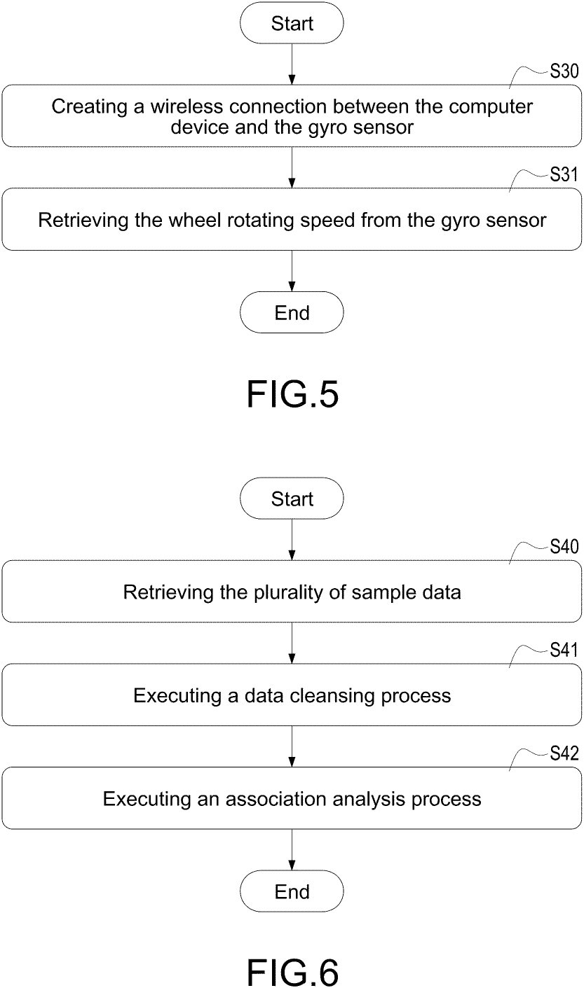

Please refers to FIG. 1 to FIG. 5. FIG. 5 is a part of a flowchart illustrating a speed generation method according to one embodiment of the present disclosure. Compared with the descriptions of FIG. 2 and FIG. 3, the speed generation method of the embodiment in FIG. 5 includes steps S30-S31 before the simulating riding service.

In step S30, the computer device 20 creates a wireless connection with the gyro sensor 21. The gyro sensor 21 is disposed on the pedals of the bicycle.

In step S31, the computer device 20 retrieves the wheel rotating speed of the bicycle from the gyro sensor 21 through the wireless connection.

Please refers to FIG. 1 to FIG. 6. FIG. 6 is a part of a flowchart illustrating a speed generation method according to one embodiment of the present disclosure. Compared with the speed generation method of FIG. 2 and FIG. 3, the speed generation method in FIG. 6 includes steps S40-S42 for generating the plurality of preset gear ratios 30.

In step S40, the cloud server 22 retrieves a plurality of sample data.

In one embodiment, each of the sample data may be retrieved from a plurality of riding records from the same or different user(s). Each of the sample data includes a sample weight (such as the riding weight records), a sample road condition (such as the road condition record), and a sample gear ratio (such as the selected gear ratio record).

In one embodiment, each of the sample data may be retrieved from the plurality of riding records of the user’s ridings in real circumstances.

In one embodiment, each of the sample data may be retrieved from the plurality of riding records of the simulating riding service provided by the smart trainer 11.

In step S41, the cloud server 22 may execute a data cleansing process to the plurality of sample data to filter the sample data determined to be an outlier.

In one embodiment, one purpose of the data cleansing process is to standardize the contents of the big data (such as the plurality of sample data) and to prevent the outlier points (or abnormal values) of the big data from influencing the analyzing result.

In one embodiment, the data cleansing process may find outlier points by a regression analysis, a statistic analysis, or a pattern distribution, and filter the sample data determined to be the outlier in big data.

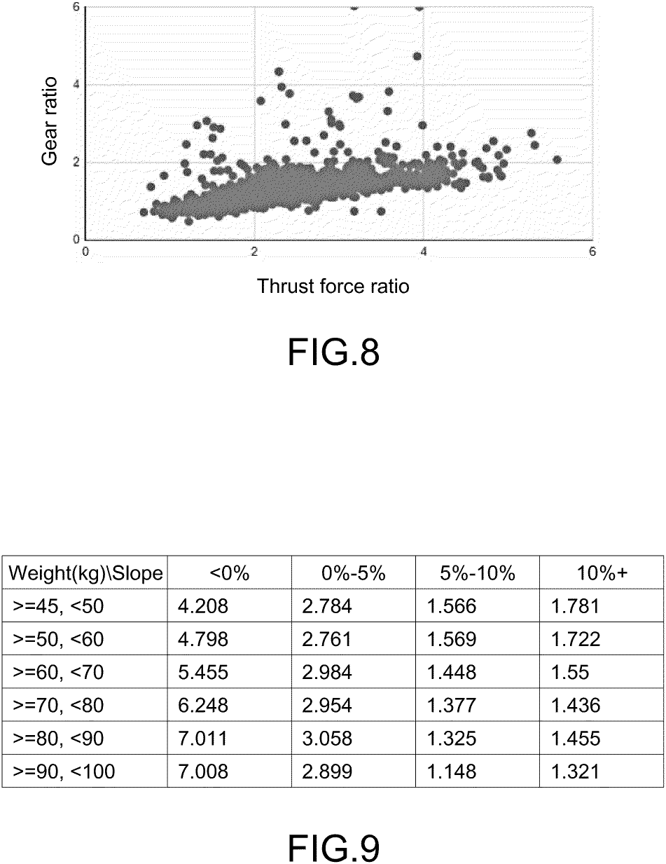

In one embodiment, please refers to FIG. 1 to FIG. 8. FIG. 8 is a diagram illustrating a distribution of a gear ratio and a thrust force ratio according to one embodiment of the present disclosure

The cloud server 22 may generate a distribution graph of the gear ratio and the thrust force ratio (the factors of the distribution graph may include the road condition and the weight) shown in FIG. 8 and remove data points with general values that do not satisfy the average value 3∂ based on the 3∂ principle to eliminate records with low probabilities.

In step S42, the cloud server 22 may execute an association analysis process to the big data (such as the plurality of sample data) after the data cleansing process and create a corresponding relation among the plurality of preset gear ratios, the plurality of weight conditions, and the plurality of road conditions.

In one embodiment, please refers to FIG. 1 to FIG. 9. FIG. 9 is a diagram illustrating a gear-ratio lookup table according to one embodiment of the present disclosure. The cloud server 22 may generate the lookup table of the preset gear ratios as shown in FIG. 9 based on the corresponding relation as discussed above, so that the lookup table may be used in following fast searching for the suitable preset gear ratio.

In one embodiment, the association analysis process may include at least one of the regression analyses (such as the linear regression and the multivariable system analysis, etc.), the statistic analysis (such as the partial correlation analysis and the variance analysis, etc.), and the machine learning algorithm (such as the deep learning, the convolutional neural network, and the shallow learning, etc.).

In one embodiment, steps S40-S42 are continually executed to keep updating the corresponding relation based on new sample data. The corresponding relation will approach a reasonable result along with the number of the sample data increasing.

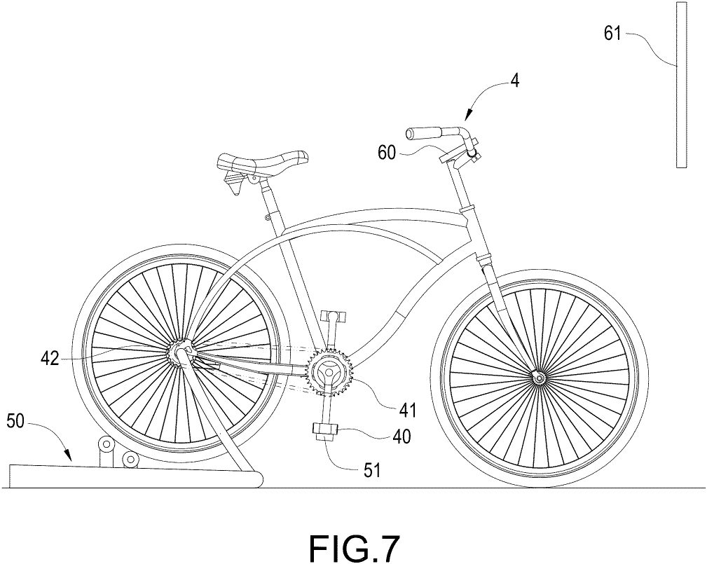

Please refers to FIG. 1 to FIG. 7. FIG. 7 is a schematic diagram illustrating the simulating riding system according to one embodiment of the present disclosure. FIG. 7 is one embodiment for illustrating an implementation in the present disclosure.

Before the simulating riding service starts, the user may dispose the gyro sensor 51 on the pedal 40 of the bicycle 4 and dispose the bicycle 4 on a bicycle frame 50.

The bicycle frame 50 is utilized for the rear wheel of the bicycle 4 to be off the ground, so the bicycle may be ridden without moving forward. When the bicycle frame 50 is utilized, it is unnecessary for the user to dismantle the freewheel 42 of the bicycle 4, and the installation time and complication are decreased.

Furthermore, the user may establish the wireless connection between the gyro sensor 51 and a smartphone 60, and the smartphone 60 may retrieve the wheel rotating speed from the gyro sensor 51 through the wireless connection.

Furthermore, the user may operate the smartphone 60 to initialize the simulating riding service, such as setting the riding weight, the tire parameter, and the riding route.

Furthermore, the display screen of the smartphone 60 may be projected onto the display 61 with a larger display size than the display screen of the smartphone 60, so the user may have better visual experience.

Furthermore, when the simulating riding service is executed, the user may push his/her foot on the pedals for driving the chainring 41, and the gyro sensor 51 provides the wheel rotating speed to the smartphone 60 in real time.

On the other hand, when executing the simulating riding service, the smartphone 60 continually executes the speed generation method to retrieve the real-time simulated speed and controls the movement of an avatar and/or a bicycle shown in the simulated screen based on the simulated speed, so the riding is simulated for the user as if the user rides in real circumstances.

It should be noted that when the road condition information changes (such as encountering an upslope) in the simulating riding service, the selected gear ratio changes accordingly (such as switching the selected gear ratio to other gear ratio for easily pedaling), so the ratio between the cycling speed of the user and the simulated speed changes accordingly (such as the cycling speed staying unchanged while the simulated speed decreasing). Therefore, the user may experience the variant speed as if he or she rides in real circumstances.

As the skilled person will appreciate, various changes and modifications can be made to the described embodiment. It is intended to include all such variations, modifications, and equivalents which fall within the scope of the present disclosure, as defined in the accompanying claims.

What is claimed is:

1. A speed generation method for simulating riding, comprising:
a) retrieving, by a computer device (20), a riding weight and road condition information of a simulating riding service;
b) selecting one of a plurality of preset gear ratios (30) as a selected gear ratio based on the riding weight and the road condition information, wherein the plurality of preset gear ratios (30) respectively correspond to a plurality of weight conditions and a plurality of road conditions;
c) retrieving a wheel rotating speed of a bicycle (4); and
d) determining a simulated speed based on the selected gear ratio, a tire parameter, and the wheel rotating speed;
wherein before step b) further comprises:
f1) retrieving a plurality of sample data, and each of the plurality of the sample data comprises a sample weight, a sample road condition, and a sample gear ratio;
f2) executing a data cleansing process to the plurality of sample data to filter the sample data determined to be an outlier; and
f3) executing an association analysis process to the plurality of sample data after the data cleansing process to create a corresponding relation among the plurality of preset gear ratios (30), the plurality of weight conditions, and the plurality of road conditions.

2. The speed generation method for simulating riding of claim 1, wherein step a) comprises:
a1) retrieving the riding weight by the computer device (20), wherein the riding weight is inputted through a human-machine interface or retrieved from user data;
a2) executing the simulating riding service and selecting one of a plurality of preset routes as a riding route according to operations; and
a3) retrieving the road condition information of a current position of the bicycle (4) in the riding route of the simulating riding service.

3. The speed generation method for simulating riding of claim 1, wherein the road condition information comprises slope information, the plurality of road conditions comprises a plurality of slope ranges, and the plurality of weight conditions comprises a plurality of weight ranges;
wherein the preset gear ratio (30) selected in step b) corresponds to the weight range matching the riding weight and the slope range matching the slope information.

4. The speed generation method for simulating riding of claim 1, wherein step c) comprises:
c1) establishing a wireless connection between the computer device (20) and a gyro sensor (51) disposed on a pedal (40) of the bicycle (4); and
c2) receiving the wheel rotating speed of the bicycle (4) from the gyro sensor (51).

5. The speed generation method for simulating riding of claim 1, wherein the tire parameter comprises a tire diameter and a tire width;
wherein in step d) the simulated speed is determined based on the selected gear ratio, the tire diameter, the tire width, and the wheel rotating speed.

6. The speed generation method for simulating riding of claim 5, wherein
$$\begin{matrix}\text{the simulated speed =} \text{π} \times \left(\text{the tire diameter +} \left(2 \times \text{the tire width}\right)\right) \\ \times \left(\text{the selected gear ratio}\right) \times \text{the wheel rotating speed}\end{matrix}$$
.

7. The speed generation method for simulating riding of claim 1, wherein the riding weight is a sum of a weight of the bicycle (4) and a weight of a user.

8. The speed generation method for simulating riding of claim 1, further comprising:
e) moving a current position of the bicycle (4) in a riding route of the simulating riding service based on the simulated speed and displaying a simulated street scene of the current position.

9. The speed generation method for simulating riding of claim 1, wherein the association analysis process comprises at least one of a regression analysis, a statistic analysis, and a machine learning algorithm.
